(19) United States
(12) Patent Application Publication
(10) Pub. No.: US 2019/0033223 A1
(43) Pub. Date: Jan. 31, 2019

(54) ASSAY DEVICE

(71) Applicants: Molecular Vision Limited, London (GB); Cambridge Display Technology Limited, Cambridgeshire (GB)

(72) Inventors: Chris Hand, York (GB); Oliver Hofmann, London (GB); Gihan Ryu, North Yorkshire (GB); Miguel Ramon, Cambridgeshire (GB); Philip Benzie, Cambridgeshire (GB); Christopher John Kay, Cambridgeshire (GB)

(73) Assignees: Molecular Vision Limited, London (GB); Cambridge Display Technology Limited, Cambridgeshire (GB)

(21) Appl. No.: 15/752,706

(22) PCT Filed: Aug. 19, 2016
(86) PCT No.: PCT/GB2016/052586

§ 371 Date: Feb. 14, 2018

(30) Foreign Application Priority Data
Aug. 19, 2015 (GB) 1514730.9

(51) Int. Cl. G01N 21/84; B01L 3/00; G01N 33/558; G01N 21/64

(52) U.S. Cl. CPC G01N 21/8483; B01L 3/5023; G01N 33/558; G01N 21/6428; G01N 2021/6439; B01L 2300/0654; B01L 2300/0825; B01L 2300/0887; B01L 2400/0406; G01N 21/6454

(57) ABSTRACT

An assay device for the quantitative determination of the concentration of at least one analyte in a liquid sample. The device comprises a lateral flow membrane (46) comprising a plurality of test regions (41A, 41B) and formed from a light transmissive material, a plurality (44A, 44B) of planar organic light emitting diode (OLED) emitters comprising an emission layer of an organic electroluminescent material, and a plurality (49A, 49B) of planar organic photodetectors (OPDs) comprising an absorption layer of an organic photovoltaic material. Each test region comprises an immobilised component for retaining analyte tagging particles. Each test region is aligned with the emission layer of one emitter and the absorption layer of one photodetector. The aligned emitter, photodetector, and test region form a group such that the emitter is capable of illuminating the test region and the photodetector is capable of detecting light from the test region. For each group, under conditions where the test region is wet and devoid of tagging particles the energised photodetector photocurrent that is produced is denoted i1, when the group emitter is the only emitter that is energised, and T2 when the emitter of another group is additionally energised. Cross-talk (C), is then represented by the black arrows, and is defined according to the equation: C=20 log₁₀ (i₁/(i₂−i₁)) C is arranged to be greater than about 20 dB for at least one group of the device.

The present invention relates to an improved assay device for the quantitative determination of the concentration of at least one analyte in a liquid sample. The liquid sample may be an original biological sample, e.g. plasma, serum, urine or saliva, or a biological sample reduced to a liquid, e.g. a plant or tissue extract.

### BACKGROUND

Chromatographic-based assay devices such as lateral flow devices (LFDs) have considerable use. One application is in devices that analyse a liquid sample to determine the presence or absence of one or more target analytes. In such devices, there may be a threshold concentration that, when exceeded, produces a qualitative indication that the analyte is present.

LFDs may also provide a quantitative indication of analyte concentration in a sample. Such devices may comprise optical measuring components to quantify a colorimetric reaction or binding event, e.g. the binding of a dye-labelled antibody/analyte complex to a second antibody immobilised on a nitrocellulose membrane.

Several techniques have been developed for optical measurement of the concentration of the target analyte, for example using light receptors coupled with a light source. Within this field, there are two broad configurations. One detects reflected emission from the light source. In this configuration, both the light source and the light detector are provided on the same side of the lateral flow membrane. Another configuration positions the light source and the light detector on opposite sides of the lateral flow membrane, such that the light or other electromagnetic radiation is transmitted through the membrane. Techniques for optical measurement of the concentration of the target analyte may include absorption or fluorescence measurements.

Inorganic LEDs and inorganic photodiodes or phototransistors may be used as the emitters and detectors for optical detection. Inorganic LEDs generally require diffusers, lenses, or other light conditioning means, to provide an even area light source suitable for measuring absorbance or fluorescence changes of an analyte test region on a nitrocellulose strip. In addition, a narrow bandwidth optical filter or other means may be needed to adjust the spectrum of the LED emitted light to better match the optical properties of the substance to be detected, or to better match the spectral sensitivity of the inorganic photodiode or phototransistor to the optical properties of the substance to be detected. The banded electronic structure of inorganic semiconductors typically provides photodiodes with broad optical absorption, therefore tailoring their spectral response generally requires the use of optical filters. The use of inorganic optoelectrical components may therefore necessitate additional expense, bulk, and decreased portability associated with these additional optical components.

Organic electroluminescent devices (OLEDs) and organic photodiodes (OPDs) may be advantageously used in the detection systems of lateral flow devices. In contrast to inorganic light emitting and detecting devices, the materials and configuration of the active layers of OLEDs and OPDs may be selected to tune the emission and absorption spectra of these devices, respectively, over a considerable wavelength range.

OLEDs and OPDs are typically planar devices that exhibit near uniform emission and absorption, respectively, across their active areas and so lenses and diffusers are not required to achieve uniform illumination of, and detection from, a LFD test region. Planar OLEDs and OPDs are also suitable for close placement parallel to a LFD strip without the requirement for intervening lenses, diffusers, optical filters, and the like.

With respect to viewing angle, OLEDs typically exhibit near-Lambertian emission profiles. In Lambertian emission the intensity of the emission varies as the cosine of the angle normal to the plane of the OLED such that intensity appears to be independent of viewing angle. OPDs likewise typically exhibit near-Lambertian absorption profiles.

The chromatographic membrane of an LFD may comprise more than one test line or test region for determining the presence or absence of one or more analyses. It may further comprise one or more control lines for determining whether or not the LFD is operating correctly. A plurality of emitters and detectors may be used for detection and more than one may be operated simultaneously. A detector may therefore detect light both from its paired emitter and also from adjacent emitters that are operated simultaneously, or from ambient light that may have entered the LFD enclosure from the exterior. The detector electrical response may thereby include an unwanted stray-light contribution. Such stray-light creates “cross-talk” between detectors that reduces the sensitivity or specificity of LFD measurements and reduces the accuracy of the test results.

WO 2005/111579 discloses a transmission-based luminescent detection system using a dispersion-type electroluminescent device comprising phosphor particles in a capacitor structure that emits light when a varying electrical field is applied to the electrodes. The publication teaches the use of opal glass or other diffusers for obtaining a near-Lambertian source. The publication does not disclose quantification of stray-light and cross-talk, how the device structure may be adapted to reduce cross-talk below a pre-defined limit, how to obtain this by adapting the source or detector, or the benefits in doing so.

Therefore there remains a need in the art for improved assay devices comprising OLEDs and OPDs with reduced stray-light and cross-talk in order to realise smaller, denser, more robust, sensitive, and accurate chromatographic-based assay devices such as LFD devices.

The present invention addresses the abovementioned shortcomings of the prior art by providing improved LFD devices.

### BRIEF SUMMARY OF THE DISCLOSURE

In accordance with the present invention there is provided an assay device for the quantitative determination of the concentration of at least one analyte in a liquid sample. The device comprises a lateral flow membrane formed from a light transmissive material and comprising a plurality of test regions, a plurality of planar organic light emitting diode (OLED) emitters comprising an emission layer of an organic electroluminescent material, a plurality of planar organic photodetectors (OPDs) comprising an absorption layer of an organic photovoltaic material, a conjugate pad in fluid communication with a proximal end of the lateral flow membrane the conjugate pad comprising optically detectable tagging particles bound to a first assay component, and a wicking pad in fluid communication with a distal end of the lateral flow membrane. The lateral flow membrane is capable of transporting a fluid from the conjugate pad to the wicking pad by capillary action. Each test region comprises an immobilised second assay component for retaining the tagging particles in the test region in dependence on the binding between the analyte, the first assay component, and the second assay component in order to generate a concentration of tagging particles in the test region that is indicative of the concentration of the analyte in the liquid sample. Each test region is aligned with the emission layer of one emitter and the absorption layer of one photodetector. The aligned emitter, photodetector, and test region form a group such that the emitter is capable of illuminating the test region and the photodetector is capable of detecting light from the test region. For each group, under conditions where the test region is wet and devoid of tagging particles and when the group emitter is the only emitter that is energised, the energised photodetector photocurrent that is produced is denoted i₁. When the group emitter and one other emitter are the only energised emitters, the energised photodetector photocurrent that is produced is denoted i₂. Cross-talk (C) is defined according to the equation:

C=20 log₁₀/(i₁/(i₂−i₁))

and C greater than about 20 dB for at least one group of the device.

Thus, in accordance with the invention, the assay device provides a relatively simple construction that is capable of determining the result of an assay by optical measurement of the test region with low cross-talk. Low cross-talk permits analyte measurements to be made with increased accuracy and with less interference from other emitters in the device. In some embodiments this improvement over the prior art enables a smaller device with a higher density of test regions. In other embodiments, it enables more groups to be included in the device, whereby more analytes may be measured or analytes may be measured using more than one test region for improved accuracy or increased assay range.

Embodiments of the invention are capable of accurately determining the concentration of an analyte in a sample. However, it is not necessary in every embodiment of the invention for the device to determine the exact concentration of the analyte. For example, in some embodiments only a qualitative indication of the analyte concentration may be determined. Typically, however, embodiments of the invention provide more than a simple yes/no indication of the presence of the analyte.

The cross-talk (C) of the present device is measured in decibels, which quantifies the extent to which light from another group emitter (or ambient light) contributes to the light detected by the photodiode of a subject group. Higher values of C correspond to less cross-talk. In devices according to the present invention, C of at least one group is greater than about 20 dB, preferably greater than about 30 dB, more preferably greater than about 40 dB, and most preferably greater than about 50 dB.

In some embodiments, and without thereby being limited by theory, the improved cross-talk of the device according to the present invention may be achieved using an emitter or photodiode that is substantially sub-Lambertian as defined herein, or both. The substantially sub-Lambertian property of the emitter or photodiode reduces the amount of light emitted or detected, respectively, at high angles as measured normal to the plane of the emitter or photodetector, which reduces cross-talk by suppressing large angle emission that may otherwise escape and interfere with detection in another group.

The present invention enables devices with an increased number of test regions. Accordingly, a device according to the present invention may have 7 or more groups, preferably 14 or more groups, and most preferably 21 or more groups.

An emitter or photodiode of the device according to some embodiments of the present invention comprise, without limitation, a distributed Bragg reflector, a strong microcavity, a substrate diffractive optical element, or a micro-lens array, in order to provide substantially sub-Lambertian emission or detection.

In another embodiment of the invention, the tagging particles absorb light at a wavelength emitted by the emitter, and the detector is arranged to detect light from the emitter passing through the lateral flow membrane, whereby the attenuation of the light intensity detected by the detector due to absorption by the immobilised tagging particles is indicative of the concentration of the analyte in the liquid sample. For example, the tagging particles may be gold nanoparticles that appear red when concentrated and may be illuminated by green light from the emitter. As a further example, the tagging particles may be blue polystyrene particles and may be illuminated by red light from the emitter. The light from the emitter may be in the visible spectrum, but may also be in the ultraviolet or infra-red wavelength ranges.

In an embodiment of the invention, the tagging particles fluoresce under illumination at a wavelength emitted by the emitter, and the detector is arranged to detect such fluorescence through the lateral flow membrane, whereby the light intensity detected by the detector due to fluorescence of the immobilised tagging particles is indicative of the concentration of the analyte in the liquid sample. For example, the tagging particles may be fluorescein or fluorescein isothiocyanate (FITC) particles illuminated with blue light.

The light transmissive material may become light transmissive when wetted by the liquid sample. The light transmissive material may be nitrocellulose. This material has been found to be particularly suitable. Dry nitrocellulose is substantially opaque. However, when wet, the nitrocellulose may become light transmissive. In this way, the nitrocellulose is particularly suitable for use in transmissive detection geometry, since light can be transmitted through the lateral flow membrane when wet. The lateral flow membrane may have a thickness of less than 200 microns.

The spacing between the facing surfaces of the emission layer and the absorption layer may be less than 1.5 mm, preferably less than 1 mm, more preferably less than 0.5 mm. Close spacing of the emission layer and the absorption layer assists in maximising the amount of captured light and therefore helps to reduce cross-talk in the device.

The emitter and/or the detector may be formed by deposition, in particular solution deposition, most particularly printing, of one or more layers on a substrate. In one embodiment, the emitter(s) and the detector(s) are provided on separate substrates. The substrate may be flexible, for example PET, or may be rigid, for example glass. In a particularly advantageous embodiment the emitter and the detector are formed on a common flexible substrate. The substrate may be folded about the lateral flow membrane. By depositing both the emitter and the detector on the same substrate correct relative alignment of the emitter and the detector can be ensured.

Typically, the emission layer comprises an organic electroluminescent material, such as an electroluminescent polymer including fluorene, poly(p-phenylene vinylene) or a phosphorescent emitter. The emission layer may comprise small molecules including organometallic chelates, fluorescent or phosphorescent dyes, or conjugated dendrimers. The organometallic chelate may be Alq₃ or an iridium containing chelate.

The active layer of the OPD typically comprises an organic photovoltaic material, usually comprising a donor and an acceptor. The acceptor may be a small molecule such as the fullerenes PCBM₆₀ or PCBM₇₀. The light absorbing donor many be a polymer such as a polythiophene, including poly(3-hexylthiophene) (P3HT). The absorption layer may therefore comprise a blend of organic photovoltaic polymers such as polythiophenes with organic photovoltaic small molecules such as PCBM₆₀ or PCBM₇₀.

The assay device may further comprise a sample pad in fluid communication with the conjugate pad and arranged to receive the liquid sample. The conjugate pad may perform the role of a sample pad, where no distinct sample pad is provided.

The lateral flow membrane may comprise a control region. The control region may be positioned between the test region(s) and the distal end of the lateral flow membrane, the control region may comprise an immobilised control component for retaining tagging particles in the control region and the emission layer and/or the absorption layer may comprise a discrete emission/absorption region aligned with the control region.

The first assay component may comprise a molecule which binds the analyte to the tagging particles and the second assay component may comprise a receptor for the analyte. This combination of components is useful in a sandwich assay.

The first assay component may comprise the analyte or an analogue thereof and the second assay component may comprise a receptor for the analyte. This combination of components is useful in a competitive assay. Alternatively, the first assay component comprises a receptor for the analyte and the second assay component comprises the analyte or an analogue thereof. The assay may be an immunoassay. The receptor may be an antibody which binds to the analyte or an analogue thereof.

The lateral flow membrane may be provided on a transparent substrate. The substrate may provide mechanical stability to the lateral flow membrane.

The assay device may comprise a controller arranged to receive detection signals from the detector and to process the detection signals whereby to generate data indicative of the concentration of the analyte in the sample. The controller may be provided as part of the assay device, for example within the same housing. The controller may also be arranged to control the emission of light from the emitter. The device may comprise a battery for powering the detector and the emitter. The device may be disposable.

The device may comprise an electrical interface for connection to an external reader, wherein the electrical interface is configured to connect the detector and the emitter to the external reader. In this way, the device can be provided as a disposable cartridge.

The assay device may comprise at least a second lateral flow membrane arranged in parallel with the first lateral flow membrane between the emitter and the detector.

Thus, in accordance with an embodiment of the invention, a second lateral flow membrane allows multiple assay tests to be performed in parallel. In some embodiments, the multiple assay tests may be testing for the same analyte in the same way. Alternatively, the multiple assay tests may test for different analytes. Performing assay tests in parallel prevents the mechanism of one assay test interfering with the mechanism of a second assay test.

The second lateral flow membrane may be provided on the same sheet as the first lateral flow membrane. The second lateral flow membrane may be joined to the first lateral flow membrane. Alternatively, the second lateral flow membrane may be provided separately to the first lateral flow membrane.

The wicking pad may be in fluid communication with a distal end of the first lateral flow membrane and a distal end of the second lateral flow membrane. Thus, the first lateral flow membrane and the second lateral flow membrane both connect to the same wicking pad.

The conjugate pad may be in fluid communication with a proximal end of the first lateral flow membrane and a proximal end of the second lateral flow membrane. Thus, the first lateral flow membrane and the second lateral flow membrane both connect to the same conjugate pad.

The conjugate pad may comprise optically detectable tagging particles bound to a third assay component.

The optically detectable tagging particles bound to the third assay component may be optically different to the optically detectable tagging particles bound to the first assay component. Thus, the different colours of the optically detectable tagging particles allow two tests to be run in close proximity without the spectrum-matched light required to test the result of one test interfering with the spectrum-matched detector required to test the result of the second, neighbouring test.

The assay device may comprise a second conjugate pad in fluid communication with a proximal end of the second lateral flow membrane.

The second conjugate pad may comprise optically detectable tagging particles bound to a third assay component. The second conjugate pad may comprise optically detectable tagging particles bound to the first assay component.

The optically detectable tagging particles in the second conjugate pad may be optically different to the said optically detectable tagging particles in the first conjugate pad.

Thus, the different colours of the optically detectable tagging particles allow two tests to be run in close proximity without the spectrum-matched light required to test the result of one test interfering with the spectrum-matched detector required to test the result of the second, neighbouring test.

In some embodiments, the second lateral flow membrane may comprise at least a second test region comprising an immobilised fourth assay component for retaining the tagging particles in the second test region in dependence on the binding between the analyte, the third assay component and the fourth assay component.

In some embodiments, the second lateral flow membrane may comprise at least a second test region comprising the immobilised first assay component for retaining the tagging particles in the second test region in dependence on the binding between the analyte, the first assay component and the second assay component.

The (first) lateral flow membrane may comprise at least a second test region comprising an immobilised fourth assay component for retaining the tagging particles in the second test region in dependence on the binding between the analyte, a (said) third assay component and the fourth assay component.

The emission layer may comprise a plurality of emitter pixels and a first emitter pixel may be aligned with the (first) test region of the first lateral flow membrane and a second emitter pixel may be aligned with the second test region.

The absorption layer may comprise a plurality of detector pixels and a first detector pixel may be aligned with the (first) test region of the first lateral flow membrane and a second detector pixel may be aligned with the second test region. The second test region may be provided on the first lateral flow membrane or the second lateral flow membrane.

The first emitter pixel and the second emitter pixel may be mutually spaced in the direction from the distal end to the proximal end of the lateral flow membrane.

The first detector pixel and the second detector pixel may be mutually spaced in the direction from the distal end to the proximal end of the lateral flow membrane.

The first detector pixel may be aligned with the first emitter pixel and the second detector pixel is aligned with the second emitter pixel.

Thus, the mutual spacing of the emitter and/or detector pixels minimises the amount of light from the first emitter pixel detectable in the second detector pixel or vice versa.

The pixels may be defined as discrete regions of the emission layer or the absorption layer. Alternatively, the emission layer or the absorption layer may be masked to define the pixels. However, this is not preferred.

### BRIEF DESCRIPTION OF THE DRAWINGS

Embodiments of the invention are further described hereinafter with reference to the accompanying drawings, in which:

FIG. 1A is an illustration of an assay device according to an embodiment of the present invention;

FIG. 1B is an illustration of a further view of an assay device according to the embodiment of FIG. 1A;

FIG. 2 is an illustration of an assay device according to a further embodiment of the present invention;

FIG. 3 is an illustration of a component of an embodiment of an assay device according to the present invention;

FIG. 4 is an illustration of two groups of a device according to an embodiment of the present invention showing cross-talk between the groups;

FIG. 5 is an illustration a group of an assay device according to an embodiment of the present invention comprising a mask;

FIG. 6 illustrates Lambertian, substantially sub-Lambertian, and super-Lambertian responses from an emitter or a detector of a device;

FIG. 7 illustrates near-Lambertian emission from the OLED of Example 1;

FIG. 8 is an illustration of an emitter of a device according to an embodiment of the present invention comprising a distributed Bragg reflector;

FIG. 9 is an illustration of an emitter of a device according to an embodiment of the present invention comprising a strong microcavity;

FIG. 10 is an illustration of an emitter of a device according to an embodiment of the present invention comprising a substrate diffractive optical element;

FIG. 11 is an illustration of an emitter of a device according to an embodiment of the present invention comprising a micro-lens array;

FIG. 12 is an illustration of a 1-row pixel pattern of an embodiment of an assay device according to the present invention;

FIG. 13 is an illustration of a 2-row pixel pattern of an embodiment of an assay device according to the present invention;

FIG. 14 is an illustration of a 3-row pixel pattern of an embodiment of an assay device according to the present invention; and

FIG. 15 is an illustration of a 4-row pixel pattern of an embodiment of an assay device according to the present invention.

### DETAILED DESCRIPTION

As used herein, the term “Lambertian” refers to emission by a planar emitter or to absorption by a planar detector in which the relative intensity of emission or absorption at an angle θ measured relative to emission or absorption at a line normal to the plane of the emitter or detector is given by cosine θ. “Super-Lambertian” emission or detection refers to emission or detection that deviates from Lambertian emission or detection in that emission at non-zero values of θ is enhanced. “Sub-Lambertian” emission or detection refers to emission or detection that deviates from Lambertian emission or detection in that emission or detection at non-zero values of θ is suppressed.

Conventional OLED devices may exhibit slight deviations from Lambertian emission (e.g. H. J. Peng, Y. L. Ho, X. J. Yu and H. S. Kwok, J. Appl. Phys. (2004) 96(3):1649-1654, and N. C. Greenham, R. H. Friend and Donal D. C. Bradley, Advanced Materials (1994) 6(6):491-494). Such slight deviations are herein termed “near-Lambertian” and contribute only slightly to the reduced cross-talk of the devices of the present invention. Specifically, certain embodiments of the low cross-talk devices of the present invention comprise near-Lambertian emitters or detectors, while other embodiments comprise substantially sub-Lambertian emitters or detectors. As described more fully herein, “substantially sub-Lambertian” emitters or detectors may be obtained through selection of structural aspects of the OLED or OPD including, without limitation, OLEDs or OPDs including a distributed Bragg reflector, a micro-lens array, a strong microcavity or a substrate diffractive optical element. “Substantial sub-Lambertian” emission or detection is that which substantially affects the directionality of emission or detection as described more fully below and therefore contributes substantially to reduced cross-talk of certain embodiments of the devices of the present invention.

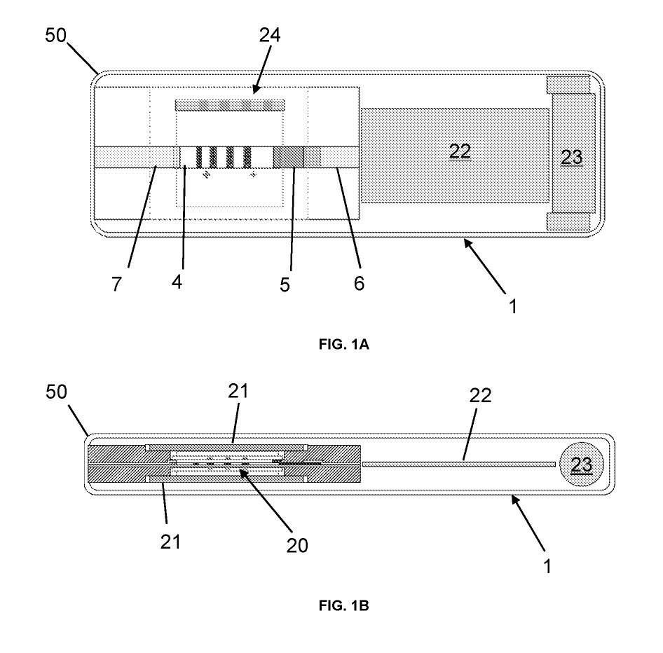

As shown in FIG. 1A and FIG. 1B, according to one embodiment of the present invention, there is provided an assay device 1 contained in a thin, substantially cuboidal housing 50, preferably constructed of an opaque material such as an opaque plastic and adapted, for example by the use of light blocking seals at any joints or ports, to minimize the entry of ambient light into the housing. FIG. 1B provides a side-on illustration of the schematic diagram for the same device as illustrated in FIG. 1A. One end of the housing contains a testing module 20 provided in the plane of the length and width of the housing 50. The opposite end of the housing 50 accommodates a cylindrical battery 23 flat against the wall of the housing 50. Between the testing module 20 and the battery 23 is a printed circuit board 22 which extends from the battery into the length of the housing in the same plane as the testing module 20. Electronics in the testing module 20 are connected to the printed circuit board 22 via an electrical interface 24. The testing module 20 contains a sample pad 6, in fluid communication with a conjugate pad 5. The present conjugate pad 5 contains particle tags which are capable of binding to an assay component. A lateral flow membrane 4 is connected between the conjugate pad 5 and a wicking pad 7. A support structure 21 secures the testing module 20 in the housing 50.

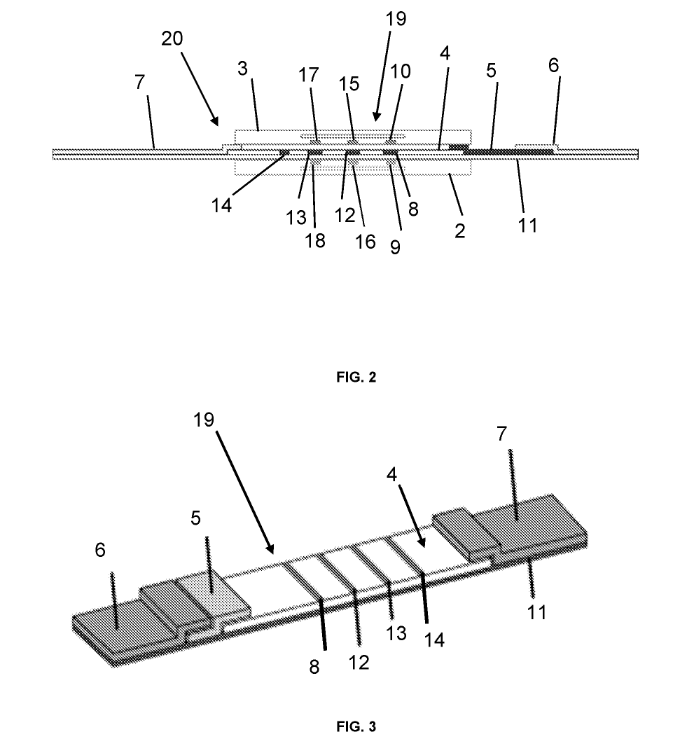

FIG. 2 illustrates a testing module 20 according to an embodiment of the present invention. The testing module according to the present invention is adapted, through selection of materials, their dimensions and relative positions, to minimize the degree of cross-talk between light emitters and detectors that are not a part of the same group, as described below. When a sample is deposited on the sample pad 6, a reservoir of excess sample is formed. The excess sample migrates to the conjugate pad 5. This migration is first caused by the conjugate pad 5, then the wicking action of the lateral flow membrane 4 and then additionally the wicking pad 7. The lateral flow membrane 4 is formed from nitrocellulose. The conjugate pad 5 contains analyte tags. The analyte tags bind to the corresponding available analyte. Capillary action causes the liquid sample, containing any tagged analytes, to flow down the lateral flow membrane 4 from the conjugate pad 5 into the testing area 19 towards the wicking pad 7. Before the sample reaches the wicking pad 7, it encounters a reaction line 8 containing fixed receptors for the analyte. When the tagged analyte reaches this point, the receptors bind to the analyte, holding the analyte and the tags in place. The presence of the coloured analyte tag will cause the reaction line 8 to change colour as the concentration of the tags increases. In the presently described example, the concentration of the coloured tags is a direct indicator of the concentration of analyte at the reaction line which provides an indication of the concentration of the analyte in the liquid sample.

The above is an example of a sandwich assay technique. A competitive assay is also possible in which the intensity of the response from the reaction line 12 (usually a colour) is inversely proportional to the amount of analyte present in the sample. In one example of this technique, the conjugate pad 5 additionally contains a pre-tagged second analyte or analyte analogue. The analyte from the sample passes unchanged through the conjugate pad 5, and will bind to the receptors on a further reaction line 12, occupying receptor sites to which the pre-tagged analytes or analyte analogues would otherwise bind. The less analyte there is in the sample, the more pre-tagged analyte or analyte analogue is able to bind to the receptors, resulting in a stronger colouring of the line. In a further example of this technique, the conjugate pad 5 could also or instead contain a tagged receptor. In this case fixed analyte or analyte analogue is immobilised on a reaction line. The more analyte present in the sample, the more of the tagged receptor that will bind to the analyte from the sample, and so not be available to bind to the fixed analyte or analyte analogue. The competitive assay technique may be used to qualitatively test for the absence of a particular analyte, though is not a purely binary test, and a very small amount of analyte in the sample is still likely to result in binding of the pre-tagged molecule (whether analyte, analyte analogue or receptor) at the position of the line. The competitive assay technique may instead be used to quantitatively indicate the concentration of a particular analyte in the liquid sample.

There is also a further line 13 of control receptors on the lateral flow membrane 4 which react with the tagged component itself. The control line 13 contains immobilised receptors which bind to the tagged component. The control line 13 should become coloured whenever the test is carried out, regardless of whether the sample contains any analyte. This helps confirm the test is performing correctly. In the presently described example, the reaction line 8 only changes colour when the analyte is present in the sample. In embodiments with multiple assays, there may be multiple control lines. In this way, the control lines can be used to determine whether each test to be performed by the lateral flow device has been performed. The control line 13 in the current example is provided downstream of the earlier reaction lines. By providing the control line 13 downstream of the reaction lines, the analyte tag must flow through the other reaction lines before they can bind to the control line indicating that a test has been carried out.

In the present case, the lateral flow membrane 4 is approximately 100 μm thick and the reaction lines 8, 12 and control line 13 are each 1.0 mm×5.0 mm, more preferably 1.0×3.0 mm, with a 2.0 mm gap between them. The lateral flow membrane is preferably formed from nitrocellulose. The sample pad 6, conjugate pad 5, lateral flow membrane 4 and wicking pad 7 are provided on a transparent substrate 11.

A reference line 14 is provided on the lateral flow membrane 4 and is used for alignment during construction of the testing area 19. The reference line 14 is typically thinner than the reaction lines 8, 12 or control line 13. The reference line in the current example is 0.5 mm×5.0 mm, more preferably 0.5 mm×3 mm, with a 1.5 mm gap between the control line 13.

Whilst the examples disclose analysing the presence, absence, or concentration of a range of analytes in the sample, it is possible to perform this analysis with fewer or more analyte tests. A range of different tags and receptor lines can be used to determine the presence, absence, or concentration of multiple different analytes. The presence of some analytes may be tested in combination with the absence of different, or the same, analytes. Tests for example assays are given in Table 1 below. In each case, the purpose of the test is given, along with the first assay component, second assay component, the analyte of interest, and which type of assay (sandwich or competitive). All assays can be performed using analyte or antibodies to the analyte labelled with any type of labelling particle. Example labelling particles include gold nano-particles, coloured latex particles, or fluorescent labels. As can be readily identified from the table in row N, assays for other analytes can be constructed using analyte antigens as the first component and antibodies to the analyte as the second component where the assay type is sandwich. Where the assay type is competitive (row M), the antibodies to the analyte would be the first component, and the analyte antigen would be the second component.

TABLE 1

| | | | | Immobilised Line | | Assay Type |
|---|---|---|---|---|---|---|
| | | | Label Binder (first | (second | | (Sandwich/ |
| | Test for: | Label | component) | component) | Analyte | Competitive) |
| A | Myeloma | All | Antibodies to free | Kappa FLC | Kappa FLC | Competitive |
| | | | kappa light chains | antigen | | |
| | | | (k-FLC) | | | |
| B | Myeloma | All | Antibodies to free | Lambda FLC | Lambda FLC | Competitive |
| | | | lambda light | antigen | | |
| | | | chains (l-FLC) | | | |
| C | Myeloma | All | Antibodies to free | Antibodies to free | Kappa FLC | Sandwich |
| | | | kappa light chains | kappa light chains | | |
| | | | (k-FLC) | (k-FLC) | | |
| D | Myeloma | All | Antibodies to free | Antibodies to free | Lambda FLC | Sandwich |
| | | | lambda light | lambda light | | |
| | | | chains (l-FLC) | chains (l-FLC) | | |
| E | Opiates | All | Antibodies to | Opiates antigen | Opiates | Competitive |
| | | | Opiates | | | |
| F | Amphetamines | All | Antibodies to | Amphetamines | Amphetamines | Competitive |
| | | | Amphetamines | antigen | | |
| G | Benzodiazepines | All | Antibodies to | Benzodiazepines | Benzodiazepines | Competitive |
| | | | Benzodiazepines | antigen | | |
| H | Cannabis | All | Antibodies to | Cannabinoid | Cannabis | Competitive |
| | | | Cannabinoids | derivative antigen | | |
| I | Cocaine | All | Antibodies to | Cocainoids | Cocaine | Competitive |
| | | | Cocainoids | antigen | | |
| J | Methamphetamine | All | Antibodies to | Methamphetamine | Methamphetamine | Competitive |
| | | | Methamphetamine | antigen | | |
| K | Methadone | All | Antibodies to | Methadone | Methadone | Competitive |
| | | | Methadone | antigen | | |
| L | Phencyclidine | All | Antibodies to | Phencyclidine | Phencyclidine | Competitive |
| | (PCP) | | Phencyclidine | (PCP) antigen | (PCP) | |
| | | | (PCP) | | | |
| M | Others | All | Antibodies to | Others antigen | Others | Competitive |
| | | | Others | | | |
| N | Others | All | Antibodies to | Antibodies to | Others | Sandwich |
| | | | Others | Others | | |
| O | Troponin I | All | Antibodies to | Antibodies to | Troponin I | Sandwich |
| | | | Troponin I | Troponin I | | |
| P | Myoglobin | All | Antibodies to | Antibodies to | Myoglobin | Sandwich |
| | | | Myoglobin | Myoglobin | | |
| Q | CKMB | All | Antibodies to | Antibodies to | CKMB | Sandwich |
| | | | CKMB | CKMB | | |
| R | Cortisol in saliva, | All | Antibodies to | Cortisol antigen | Cortisol | Competitive |
| | serum or urine | | Cortisol | | | |

Whilst common household assay tests, such as some pregnancy tests, have an apparently binary result and require a user to manually interpret the results, the present device uses an Organic Light Emitting Diode (OLED) and opposed Organic Photo Diode (OPD) to measure the light absorption as a result of the analyte test. Whilst the presently described embodiment uses the absorption of light by a substance to indicate the concentration of an analyte in a test sample, embodiments can equally be envisaged where the tag on the analyte is luminescent and emits light itself, either as a result of fluorescence, phosphorescence, or as a result of a chemical or electrochemical reaction.

The assays for Myeloma are described in rows labelled A-D in Table 1. To test for myeloma, the ratio of Kappa FLC concentration to Lambda FLC concentration is determined.

The OLED illuminates the sample with light having known characteristics (intensity, wavelength, etc). When light is received by the OPD, a current is generated. By measuring this current (e.g. directly or as a voltage following amplification), the light absorbed by the immobilised labels at the reaction line, 8, 12 and surrounding membrane can be determined. This gives an indication of the concentration of tagged analyte present in the sample.

The OLED is formed as a layered structure supported on a substrate, and comprises an anode, cathode and a light-emitting layer between the anode and cathode. The substrate can be flexible or rigid. Suitable substrate materials include, without limitation, plastics such as PET, glass, or a laminated structure comprising one or more alternating plastic and inorganic barrier layers. One or more further layers may be provided between the anode and the cathode, for example to aid charge injection, charge transport, or charge balance. Optionally, further layers may be selected from one or more of a hole-injection layer, a hole-transporting layer, an electron-blocking layer, an electron-transporting layer, and a triplet blocking layer.

Exemplary OLED layer structures include the following:

Anode/Light-emitting layer/Cathode
Anode/Hole transporting layer/Light-emitting layer/Cathode
Anode/Hole-injection layer/Hole-transporting layer/Light-emitting layer/Cathode
Anode/Hole-injection layer/Hole-transporting layer/Light-emitting layer/Electron-transporting layer/Cathode.
Preferably, a hole-injection layer is present between the anode and the light-emitting layer.
Preferably, a hole-transporting layer is present between the anode and the light-emitting layer.
Preferably, both of a hole-injection layer and a hole-transporting layer are present.

In one embodiment, substantially all light is emitted from the primary light-emitting layer. In other embodiments, one or more further layers may also emit light. Optionally, one of a hole-transporting layer and an electron-transporting layer comprises a light-emitting material and emits light in use.

In some embodiments, the OLED may be formed from a layer of patterned ITO (indium tin oxide, which is conductive and transparent), a layer of hole injection material, a layer of active light-emitting material, and a cathode. Referring now to FIG. 2, substrate 2 contains OLED emission regions 9, 16, 18, provided opposite the organic photovoltaic cell substrate 3, containing detection regions 10, 15, 17. The emission light colour of all three regions in the present example is blue, as they are formed from a layer of the same material. Similarly, in the present example, the material of the detection regions 10, 15, 17 is optimised to detect blue light.

The areas of the OLED emission regions, OPD detection regions, LFD test region, the aperture sizes of an optional intervening opaque mask, and the separation of the mask and OLED and mask and OPD are selected to reduce cross-talk from other OLED emission regions.

Emission regions 9, 16, 18 and detection regions 10, 15, 17 are sized to sit within the footprint of reaction lines 8, 12, 13, 14 containing bound receptors set up to catch and bind the tagged analyte (pre-tagged or otherwise). Exemplary pixel sizes include 0.9 mm×4.9 mm, 0.5 mm×2 mm, 0.5 mm×1 mm, or smaller. This maximises the proportion of the light emission from the OLED that is capable of interacting with the tagged analyte and the surrounding lateral flow membrane 4.

Another factor which improves the proportion of the emitted light that can interact with the membrane and tagged analyte, and reduce cross-talk, is the proximity of both the OLED and the OPD to the lateral flow membrane 4. This separation may be less than about 2 mm. Typically, the chromatographic membrane is supported on a substrate such as a transparent plastic layer. Because this layer may be impermeable, the OLED or OPD may be positioned closer to this side of the membrane than to the opposite side. In a preferred embodiment, the separation between OLED or OPD and the membrane support is less than 1 mm, more preferably less than 0.5 mm, and most preferably about 0.2 mm. The separation between the OLED or OPD and the opposite side of the membrane is less than 2 mm and preferably 1 mm or less.

The circuit board 22 and battery 23 included within the housing 50 for the assay device 1 control and power the OLED and OPD. The circuit board 22 also includes a microprocessor suitable for performing basic analysis in order to calculate a quantitative value representative of the amount of the analyte(s) present in the sample and/or ratios thereof.

For an example OPD the following structure can be used. The first layer (closest to the membrane) is a pre-patterned indium-tin-oxide (ITO) glass substrate. The glass substrate provides a barrier layer for the OPD. On top of the ITO layer is provided a 50 nm thick layer of Baytron P grade poly(styrenesulphonate)-doped poly(3,4-ethylenedioxythiophene) (PEDOT:PSS) and 10 nm thick Poly(methyl methacrylate) (PMMA) film interlayer is provided thereon. The active layer is 165 nm thick regioregular poly(3-hexylthiophene):1-(3-Methoxycarbonylpropyl)-1-phenyl-[6.6]061 (P3HT:PCBM) with an upper electrode for the device of 100 nm-thick aluminium.

This is only one example of an OPD suitable for use in embodiments of the present invention. The skilled person will be aware of methods of manufacturing such OPDs and other materials from which suitable OPDs may be manufactured.

The skilled person is aware of several methods and material combinations from which to fabricate OLEDs suitable for the present invention. In one particular OLED type, the structure is a plastic substrate (PET), a layer of patterned ITO, a layer of hole injection material, a layer of active material, and a cathode. In particular, the spectrum output of the OLED can be selected by the correct choice of the organic polymer or other small molecule.

The spectrum of emission of the OLED must be matched to the absorbance of the relevant light quencher (the coloured tags used to label the compound of interest). In an absorbance regime, gold nanoparticles can be used. In this case, a green illumination source should be used. Alternatively, blue polystyrene labels can be used. In this case, a red illumination source should be used. In a fluorescence regime, fluorescein/FITC based labels can be used. In this case, a blue illumination source should be used.

The present invention is directed to an assay device in which cross-talk between emitter-detector groups within the device is surprisingly reduced. A “group” as the term is used herein comprises a test region aligned with the emission layer of one emitter and the absorption layer of one photodetector. Thus, within a group the emitter is capable of illuminating the test region, and the photodetector is capable of detecting light from the test region.

Cross-talk is preferably measured when the test region is wet and devoid of tagging particles. For each group, the energised photodetector photocurrent is denoted as when the group emitter is the only energised emitter. The photodetector photocurrent may be measure directly or following amplification or other signal processing, and may be detected as a current or as a voltage representing that current. Similarly, i₂ denotes the photocurrent when the group emitter and one other emitter are the only energised emitters. Cross-talk (C) may then be defined according to the following equation:

C=20 log₁₀(i₁/i₂−i₁))

In devices according to the present invention, cross-talk is surprisingly low as reflected in a high C value of greater than about 20 dB, preferably greater than about 30 dB, more preferably greater than about 40 dB, and most preferably greater than about 50 dB, for at least one group in the device.

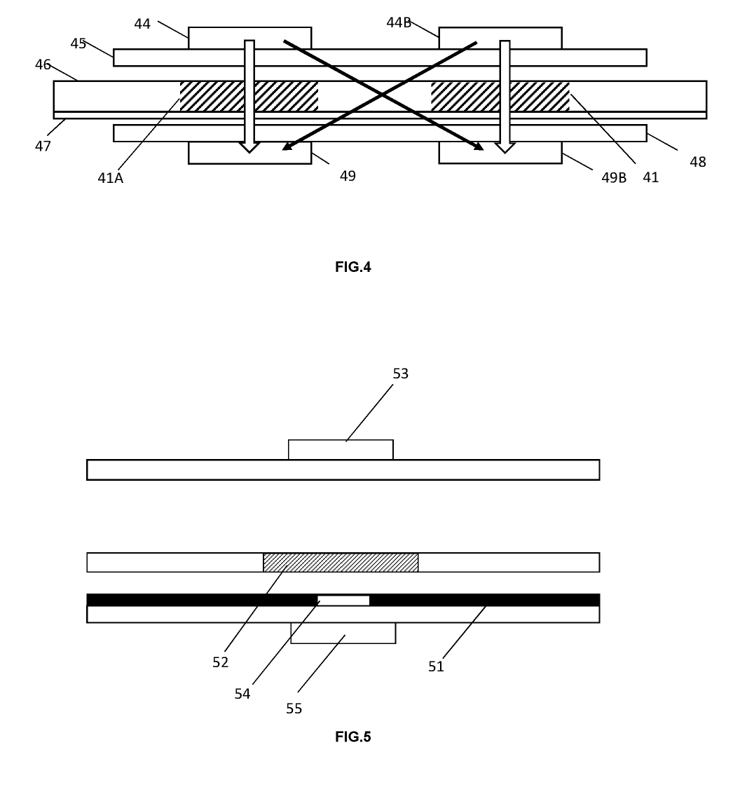

FIG. 4 illustrates cross-talk between two groups of the device. Although directly adjacent groups are shown, cross-talk may be measured between any pair of groups in the device, and between any group and ambient light entering housing 50. Group A comprises OLED 44A, OPD 49A and test region 41A, and Group B comprises OLED 44B, OPD 49B and test region 41B. Test regions 41A and 41B are within membrane 46, which is supported on transparent support 47. OLEDs 44A and 44B are supported on substrate 45, and OPDs 49A and 49B are supported on substrate 48. Open arrows denote light transmission from OLED to OPD through a test region within a group. When only one group, for example group A, is energised, the photocurrent measured corresponds to i₁. When group B is also energised, the photocurrent measured at OPD 49A will comprise contributions from the emitters of both groups A and B, and corresponds to i₂ in the above formula. The contribution of OLED 44B to the photocurrent of OPD 49A arises from cross-talk, as denoted by the solid arrow light paths. It will be readily understood that many other light paths in addition to the direct path shown may also contribute to cross-talk.

Cross-talk may be reduced by positioning an opaque mask comprising apertures between the OLED and OPD of a group. The material of the mask is not particularly limited and may, for example, be formed from an opaque plastic having a thickness of about 100 μm.

FIG. 5 shows a preferred arrangement of a mask in a group. Mask 51 is interposed between test region 52 and OPD 53. Preferably mask aperture 54 is smaller than and within the area of test region 52. OPD 53 may then be positioned to receive light from OLED 55 via the test region but preferably not from the membrane extending beyond the test region. The positions of the OLED and OPD may be reversed.

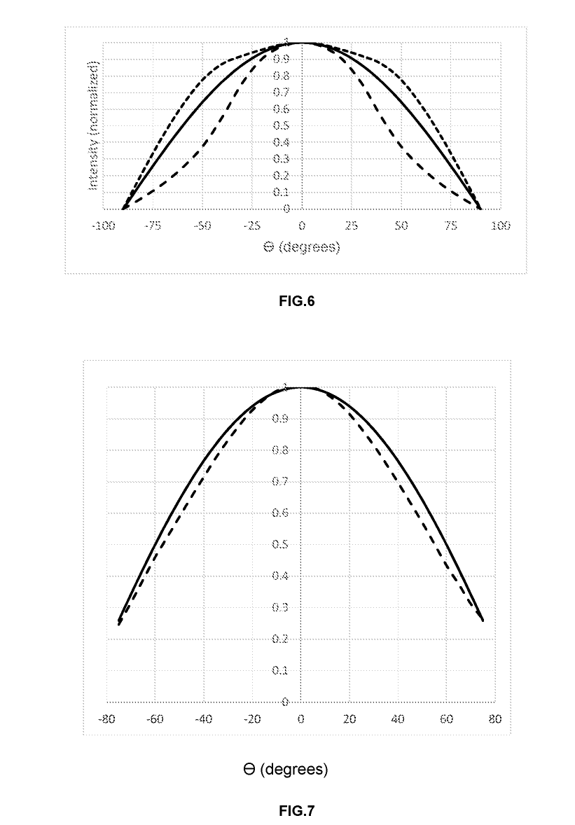

In some embodiments of the device according to the present invention, a further reduction in cross-talk is achieved using substantially sub-Lambertian emitters and/or detectors. This can be achieved using a number of OLED and OPD structures known in the art. FIG. 6 illustrates the angular dependence of emission or detection for OLEDs or OPD's, respectively, for Lambertian (solid line), super-Lambertian (short dashes), and substantially sub-Lambertian (long dashes) properties. In the substantially sub-Lambertian case, forward emission or detection is enhanced and emission or detection at large angles is suppressed. Either the emitter or detector, or both, may exhibit substantially sub-Lambertian properties. Maximising the forward emission ensures that a maximised amount of the light emitted by the OLED is emitted perpendicular to the active surface of the device. In this way, the light emitted by the OLED that passes through the light quenchers and onto the OPD is maximized. This increases both the sensitivity and accuracy of these devices.

An OLED or OPD lacking a strong microcavity or other feature giving rise to substantially sub-Lambertian properties may exhibit near-Lambertian emission or absorption. FIG. 7 shows an example of near-Lambertian emission in an OLED. Near-Lambertian emission may arise because of weak microcavity effects that arise from the relative thicknesses and refractive indices of the conventional OLED layers.

Substantially sub-Lambertian emission or detection may be achieved using a number of structures known in the art that may be incorporated into OLEDs or OPDs, respectively. FIG. 8 shows OLED 80 comprising a distributed Bragg reflector. OLED active layers 81-83 are between reflective cathode 84 and transparent anode (ITO) 85. A distributed Bragg reflector is interposed between anode 85 and substrate 86. The reflector comprises alternating transparent layers of differing refractive index. Lower refractive index layers 87A and 87B (e.g. SiO₂, n=1.5) are alternated with higher refractive index layers 88A, 88B, 88C (e.g. TiO2, n=2.45). The thicknesses are selected to produce a quarter wavelength dielectric stack, according to the emission wavelength of the OLED, and are each sufficiently thin (e.g. 50-100 nm) that the reflector does not significantly affect the placement of elements within a group. The number of alternating low and high refractive index layers can be increased to increase forward emission/detection and reduce emission/detection at large angles. U.S. Pat. No. 6,366,017, and Choy, W. C. H. and Ho, C. Y. (2007) Optics Express 15(20):13288-13294 disclose OLEDs comprising distributed Bragg reflectors and exhibiting substantially sub-Lambertian emission and are hereby incorporated by reference in their entireties.

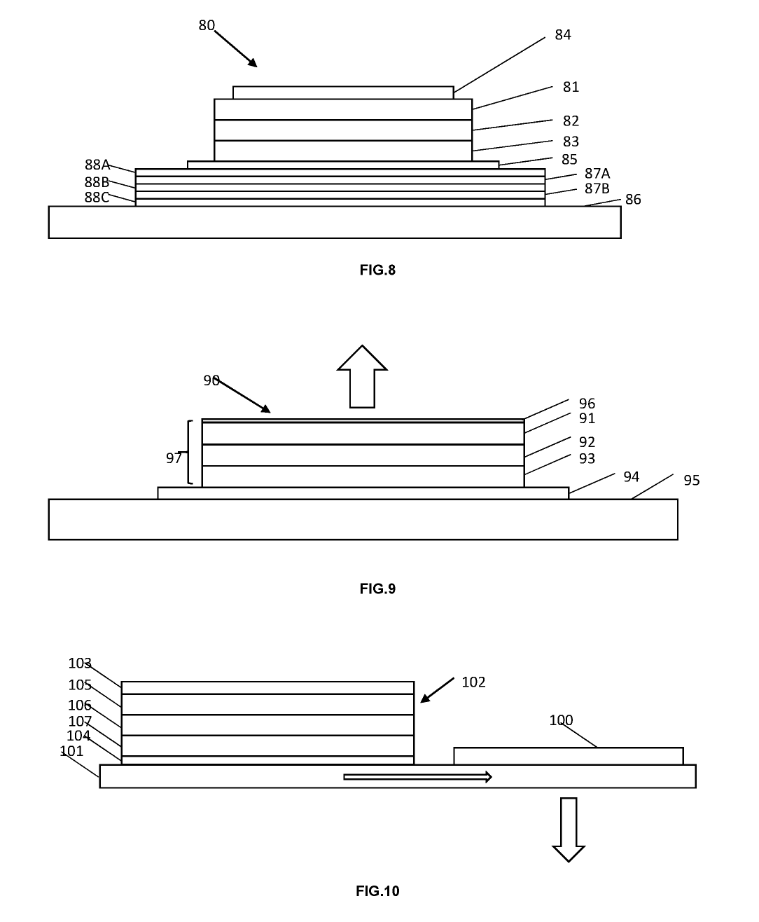

Substantially sub-Lambertian emission or detection can also be achieved using a strong microcavity. As used herein, a “strong microcavity” is formed in an OLED or OPD in which one electrode is highly reflective, e.g. Ag or Al, and one is partially reflective, e.g. thin Ag. FIG. 9 shows an OLED comprising a strong microcavity 90. OLED active layers 91, 92 and 93 are disposed between reflective electrode 94 as supported on substrate 95, and partially reflective electrode 96. The two electrodes form strong microcavity 97, and the separation of the electrodes is selected according to the wavelength of the emitted light (e.g. one-half wavelength) to maximise forward emission. The direction of emission is shown by the large arrow. Lin, C-L. and WU C-C. (2005) Appl. Phys. Lett. 87:021101-1-021101-3 discloses an OLED comprising a strong microcavity and substantially sub-Lambertian emission and is hereby incorporated in its entirety by reference.

Substantially sub-Lambertian emission or detection can also be achieved by including a diffractive optical element adjacent to an OLED or OPD. FIG. 10 shows an OLED adjacent to a diffractive optical element 100 on a common substrate 101. OLED 102 comprises reflective electrode 103 and transparent electrode 104, and active layers 105, 106 and 107 therebetween. Diffractive optical element 100 is a nanoprinted photonic structure formed, for example, from photoresist patterned by photolithography. A portion of the light emitted by the OLED forms directional substrate modes within the substrate, which are selectively extracted by diffractive optical element 100, resulting in highly directional emission, shown by the broad arrow, without Lambertian background. S. Zhang, G. A. Turnbull and Samuel, I. D. W. (2014) Adv. Optical Mater. 2:343-347 discloses an OLED adjacent a diffractive optical element on a common substrate that exhibits sub-Lambertian emission, and is hereby incorporated in its entirety by reference.

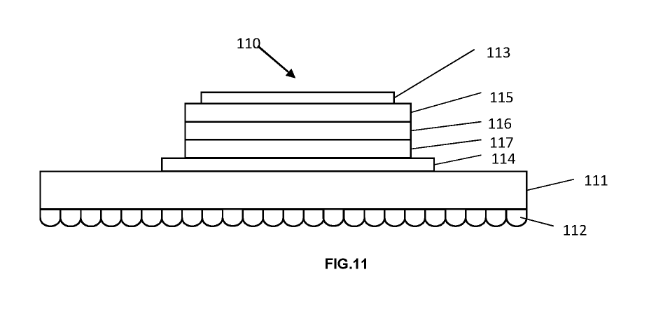

Substantially sub-Lambertian emission or detection can also be achieved using a micro-lens array. FIG. 11 (not to scale) shows an OLED 110 supported by a substrate 111 comprising a micro-lens array 112. OLED 110 comprises reflective electrode 113 and transparent electrode 114, and active layers 115, 116 and 117 therebetween. The micro-lens array can be an array of elements having a hemispherical or prism or other shape, typically each having dimensions of tens to hundreds of microns, arrayed on the substrate surface such as, for example, hemispheres fabricated on or attached to the outward surface of the substrate. Diffraction at each element reinforces to provide enhanced forward emission and substantially sub-Lambertian emission. Danz, N., Wachter, C. A., Michaelis, D. Dannberg, P. and Flammich M. (2012) Optics Express 20(12): 12682-12691 discloses OLEDs comprising micro-lens arrays and exhibiting substantially sub-Lambertian emission, and is hereby incorporated in its entirety by reference.

The present invention provides devices with low cross-talk between groups. An advantage of the present invention is that the density of groups within the device can be higher than devices of the prior art. The devices may therefore be smaller, or may contain more groups. If they contain more groups, more analytes may be measured within a device, or more measurements can be made on each analyte to increase accuracy or extend the measurement range, or both.

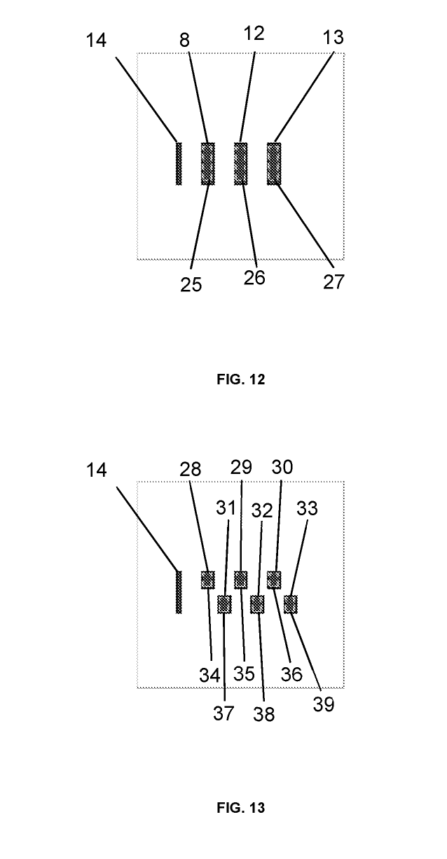

FIG. 12 illustrates a 1-row pixel pattern of an embodiment of an assay device according to the present invention. The reference line 14, reaction lines 8 and 12, and control line 13 are provided on the lateral flow membrane. The OLED and OPD production processes allow pixels of any size and positioning to be created to overlay the reaction and control lines. In FIG. 12, the pixel outlines 25, 26, and 27 shown as dashed lines represent the outline of the OPD sensitive regions and OLED pixels. These pixels are centred on the reaction lines 8, 12 (or control line 13). The pixel outlines 25, 26, and 27 are also smaller than the reaction lines 8, 12 (or control line 13). In this way, the light which enters the OPD from the OLED without passing through the reaction line (i.e. passing through a part of the lateral flow membrane not forming part of the reaction line or control line) is minimised and/or substantially eliminated. In some embodiments, the pixel outlines may have substantially the same extent as the reaction lines. The reaction lines 8, 12 may be correspond to assays for the same analyte. In this way, the accuracy of any resulting indications of the analyte concentration in the liquid sample can be maximised by multiple assays of the same sample.

FIG. 13 illustrates a 2-row pixel pattern of an embodiment of an assay device according to the present invention. In this embodiment, there are two parallel lateral flow membranes. As described previously, the reference line 14 is used to align the reaction regions 28, 29, 30, 31, 32, 33 with the OPD and OLED outlines 34, 35, 36, 37, 38, 39 respectively. By diagonally offsetting the matched reaction regions (lines) from each other, the light bleed between two neighbouring reaction regions, is minimised. In this way, for example, the amount of light from the OPD/OLED outline 37 detectable by the OPD on the OPD/OLED outline 34, 35 is minimised. This allows a particularly compact arrangement of assays in a single assay device. In some embodiments, each parallel lateral flow membrane can contain a single reaction region, with each lateral flow membrane testing for a different analyte. In other embodiments, each parallel lateral flow membrane can contain a single or multiple reaction regions, with each lateral flow membrane testing for the same one or group of analytes. This allows the accuracy of the resulting indications of the analyte concentrations in the liquid sample to be improved. In yet other embodiments, multiple testing regions on a plurality of parallel lateral flow membranes can be used to test for the same analyte in different ways. In this way, one lateral flow membrane may test for a given analyte using a sandwich assay technique, whilst another lateral flow membrane may test for the same given analyte using a competitive assay technique.

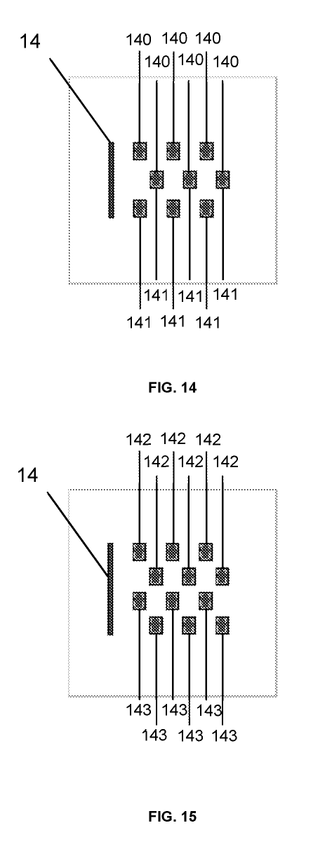

FIGS. 14 and 15 illustrate respectively a 3-row and 4-row pixel pattern of an embodiment of an assay device according to the present invention. The reaction regions 140, 142 provided on the lateral flow membrane are arranged to minimise light from the OLED having outline 141, 143 bleeding into the outline of any neighbouring OPD having outline 141, 143. As before, the reference line 14 is provided for alignment purposes.

Whilst in the embodiments shown, the reaction lines and/or reaction regions are intended to extend to each side of each lateral flow membrane, as seen specifically in reaction line 12, the invention extends to alternative embodiments where the reaction lines and/or reaction regions do not extend to each side of each lateral flow membrane. For example, the reaction regions may be centred in the middle of the lateral flow membrane. Alternatively, two distinct regions may be provided side-by-side on a lateral flow membrane. There may be a space on the lateral flow membrane between the two reaction regions. In some embodiments, the two reaction regions are provided in contact with each other. In some embodiments, two or more regions may be spaced or offset both in the proximal-distal direction, and in the width direction of the lateral flow membrane. The reaction regions may be provided on distinct lateral flow membranes which may be provided, for example, side-by-side.

Whilst embodiments of the present invention have been described using direct tagging, indirect tagging is also possible. In embodiments where a first antibody binds to the analyte, the tagging particle may be bound to a further antibody, which is configured to bind to the first antibody. In this way the same labelled antibody can be used for several different analytes.

Whilst the embodiments shown use a conjugate pad, it will be appreciated that the sample may be pre-treated with the analyte tags. This may ensure better mixing and binding between the analyte and analyte tags, particularly where there are very low concentrations of analyte. In this case, the conjugate pad is not required, and the pre-treated sample may be deposited on the sample pad or the lateral flow membrane directly. In some embodiments where the presence or concentration of multiple analytes is to be tested, the sample may be pre-treated for only some of the analytes of interest. In this case, a conjugate pad is still required.

Whilst the embodiments shown are for quantitative measurements, it will be appreciated that the invention is equally applicable to qualitative or semi-quantitative assay devices, where only a presence or absence indication of one or more analytes of interest is required. In semi-quantitative assay devices, only a discretised reading of, for example, a plurality concentration levels is required. The concentration levels need not be regularly spaced over the range of concentration to be measured.

An advantage of the present invention in embodiments using fabricated OPDs and OLEDs compared to prior art devices using silicon-based inorganic detectors or GaAs and/or InGaAs and/or SbGaInAs-based inorganic emitters is the ability to provide multiple assays (quantitative or otherwise) without a corresponding increase in material costs. In the inorganic emitters and detectors of the prior art, multiple reaction regions require multiple emitters and detectors, which each have a unit cost. In embodiments of the present invention, OPDs and OLED are fabricated from a single piece, regardless of the number of pixels the emitter or detector requires, and so there is only a minimal increase in cost for the provision of an additional reaction region.

### Example 1

A device comprising seven groups otherwise substantially as shown in FIGS. 1 and 2 was provided in which the OLED detectors were manufactured using solution processing and had the following structure:

glass/ITO/hole injection layer/polymer host+Ir-dendrimer green emitter/Ag
FIG. 7 shows the angular dependence of the emission profile of the OLED emitter (broken line) compared with Lambertian emission (solid line), showing that emission is near-Lambertian. The OPD detectors were also manufactured using solution processing and had the following structure:
glass/ITO/hole transporting layer/polymer donor+acceptor/Ag
A mask was disposed between the OLED substrate and the membrane support and the separation between them was about 0.2 mm. The OPD substrate was about 1.0 mm from the wet nitrocellulose membrane that was devoid of tagging particles. The OLED and OPD pixel sizes were 0.5 mm×2 mm, groups were spaced apart by 2 mm, and the mask aperture size was 0.5 mm×2.4 mm. The cross-talk (C) between the third and the seventh group was 21.1 dB.

### Example 2

A device otherwise substantially as in Example 1 is provided in which the OLED emitters further comprise a distributed Bragg reflector positioned between the ITO and substrate and having the structure:

[ITO, 50 nm]/TiO₂, 56 nm/SiO₂, 92 nm/TiO₂, 56 nm/SiO₂, 92 nm/TiO₂, 56 nm/[glass]
and the device comprises 21 groups. The OLEDs exhibit substantially sub-Lambertian emission. The cross-talk (C) between at least two groups is at least 30 dB.

### Example 3

A device otherwise substantially as in Example 1 is provided in which the OLED emitters are top emitting OLEDs comprising a strong microcavity and have the structure:

glass/Ag, 85 nm/hole transporting layer/polymer donor+acceptor/Ag, TeO₂, 10 nm
and the spacing between electrodes is about 250 nm. In this embodiment, the OLEDs are positioned on the side of the substrate nearer to the membrane as these OLEDs are top-emitting.
The device comprises 21 groups and the OLEDs exhibit substantially sub-Lambertian emission. The cross-talk (C) between at least two groups is at least 40 dB.

### Example 4

A device otherwise substantially as in Example 1 is provided in which the OLED emitters are each adjacent a substrate diffractive element through which emission is obtained, substantially as shown in FIG. 14. The OLEDs exhibit substantially sub-Lambertian emission with strongly directional emission. The cross-talk (C) between at least two groups is at least 50 dB.

### Example 5

A device otherwise substantially as in Example 1 is provided in which the OLED emitters further comprise a micro-lens array comprising hemispherical lenses of 200 μm diameter positioned on the emissive surface of the OLED substrate. The device comprises 21 groups. The OLEDs exhibit substantially sub-Lambertian emission. The cross-talk (C) between at least two groups is at least 40 dB.

### Example 6

A device otherwise substantially as in Example 1 is provided in which the OPD detectors further comprise a micro-lens array comprising hemispherical lenses of 200 μm diameter positioned on the surface of the OPD substrate. The device comprises 21 groups. The OPDs exhibit substantially sub-Lambertian detection. The cross-talk (C) between at least two groups is at least 40 dB.

In summary, an assay device for the quantitative determination of the concentration of at least one analyte in a liquid sample and having low cross-talk comprises a planar emitter 2, a planar detector 3, a lateral flow membrane 4 interposed between the emitter 2 and the detector 3, a conjugate pad 5 in fluid communication with a proximal end of the lateral flow membrane 4, the conjugate pad 5 comprising optically detectable tagging particles bound to a first assay component, a sample pad 6 in fluid communication with the conjugate pad 5 and arranged to receive the liquid sample, and a wicking pad 7 in fluid communication with a distal end of the lateral flow membrane 4. The lateral flow membrane 4 is formed from a light transmissive material and is capable of transporting fluid from the conjugate pad 5 to the wicking pad 7 by capillary action. The lateral flow membrane 4 comprises at least one test region 8,12 comprising an immobilised second assay component for retaining the tagging particles in the test region 8,12 in dependence on the binding between the analyte, the first assay component and the second assay component in order to generate a concentration of tagging particles in the test region 8,12 that is indicative of the concentration of the analyte in the liquid sample. The emitter 2 comprises an emission layer 9,16 of an organic electroluminescent material and the emission layer 9,16 is aligned with the test region 8,12 of the lateral flow membrane 4, whereby the emitter 2 is capable of illuminating the test region 8,12. The detector 3 comprises an absorption layer 10,15 of an organic photovoltaic material and the absorption layer 10,15 is aligned with the test region 8,12 of the lateral flow membrane 4, whereby the detector 3 is capable of detecting light from the test region 8,12. Embodiments of the present invention allow for the fabrication of fully disposable quantitative multi-zone diagnostic devices ideally suited for home testing.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. An assay device for the quantitative determination of the concentration of at least one analyte in a liquid sample, the device comprising:
a lateral flow membrane formed from a light transmissive material, the membrane comprising a plurality of test regions;
a plurality of planar organic light emitting diode (OLED) emitters comprising an emission layer of an organic electroluminescent material;
a plurality of planar organic photodetectors (OPDs) comprising an absorption layer of an organic photovoltaic material;
a conjugate pad in fluid communication with a proximal end of the lateral flow membrane, the conjugate pad comprising optically detectable tagging particles bound to a first assay component;
a wicking pad in fluid communication with a distal end of the lateral flow membrane;
wherein the lateral flow membrane is capable of transporting a fluid from the conjugate pad to the wicking pad by capillary action;
wherein each test region comprises an immobilised second assay component for retaining the tagging particles in the test region in dependence on the binding between the analyte, the first assay component and the second assay component in order to generate a concentration of tagging particles in the test region that is indicative of the concentration of the analyte in the liquid sample;
wherein each said test region is aligned with the emission layer of one said emitter and the absorption layer of one said photodetector, the aligned emitter, photodetector, and test region forming a group whereby the emitter is capable of illuminating the test region, and the photodetector is capable of detecting light from the test region;
wherein, when the test region is wet and devoid of tagging particles, for each group
the energised photodetector photocurrent is I₁ when the group emitter is the only energized emitter and is I₂ when the group emitter and one other emitter are the only energised emitters, whereby cross-talk (C) is defined according to the following equation:
C=20 log₁₀(i₁/(i₂−i₁))
and C greater than about 20 dB for at least one group.

2. The assay device as claimed in claim 1, wherein C is greater than about 30 dB for at least one group.

3. (canceled)

4. (canceled)

5. The assay device as claimed in claim 1, wherein at least one emitter is a substantially sub-Lambertian emitter and/or at least one photodetector is a substantially sub-Lambertian photodetector.

6. (canceled)

7. The assay device as claimed in claim 1, wherein the number of groups is 7 or more.

8. (canceled)

9. (canceled)

10. The assay device as claimed in claim 5, wherein the substantially sub-Lambertian emitter and/or the substantially sub-Lambertian photodetector comprises a distributed Bragg reflector, a strong microcavity, a substrate diffractive optical element, or a micro-lens array.

11. (canceled)

12. (canceled)

13. (canceled)

14. (canceled)

15. (canceled)

16. (canceled)

17. (canceled)

18. (canceled)

19. The assay device as claimed in claim 1, wherein the tagging particles fluoresce under illumination at a wavelength emitted by the emitter, and the detector is arranged to detect such fluorescence through the lateral flow membrane, whereby the light intensity detected by the detector due to fluorescence of the immobilised tagging particles is indicative of the concentration of the analyte in the liquid sample.

20. The assay device as claimed in claim 1, wherein the light transmissive material is nitrocellulose.

21. (canceled)

22. (canceled)

23. The assay device as claimed in claim 1, wherein the spacing between the facing surfaces of the emission layer and the lateral flow membrane and/or between the absorption layer and the lateral flow membrane is less than 1 mm.

24. (canceled)

25. (canceled)

26. The assay device as claimed in claim 1, wherein the emitter and the detector are formed on a common substrate, which is folded about the lateral flow membrane.

27. An assay device as claimed in claim 1, wherein the emission layer comprises an organic electroluminescent polymer.

28. An assay device as claimed in claim 1, wherein the absorption layer comprises an organic photovoltaic polymer.

29. An assay device as claimed in claim 1, wherein the lateral flow membrane comprises a control region between the test region(s) and the distal end of the lateral flow membrane, the control region comprising an immobilised control component for retaining tagging particles in the control region and the emission layer and/or the absorption layer comprises a discrete emission/absorption region (pixel) aligned with the control region.

30. An assay device as claimed in of claim 1, wherein the first assay component comprises a molecule which binds the analyte to the tagging particles and the second assay component comprises a receptor for the analyte.

31. An assay device as claimed in claim 1, wherein the lateral flow membrane is provided on a transparent substrate.

32. An assay device as claimed in claim 1, further comprising a controller arranged to receive detection signals from the detector and to process the detection signals whereby to generate data indicative of the concentration of the analyte in the sample.

33. An assay device as claimed in claim 32, wherein the controller is arranged to control the emission of light from the emitter.

34. An assay device as claimed in claim 1, further comprising a battery for powering the detector and the emitter.

35. An assay device as claimed in claim 1, further comprising an electrical interface for connection to an external reader, wherein the electrical interface is configured to connect the detector and the emitter to the external reader.

36. An assay device as claimed in claim 1, wherein the device is disposable.

37. An assay device as claimed in claim 1, comprising at least a second lateral flow membrane arranged in parallel with the first lateral flow membrane.
